(19) United States
(12) Patent Application Publication
(10) Pub. No.: US 2022/0001027 A1
(43) Pub. Date: Jan. 6, 2022

(54) Genetically Engineered Skin Cells for the Systemic In Vivo Treatment of Deficient Enzymes, Factors or Proteins

(71) Applicants: President and Fellows of Harvard College, Cambridge, MA (US); Massachusetts Institute of Technology, Cambridge, MA (US); The General Hospital Corporation, Boston, MA (US)

(72) Inventors: Denitsa M. Milanova, Boston, MA (US); George M. Church, Brookline, MA (US); Isaac Han, Somerville, MA (US); James Gorman, Wellesley, MA (US); Robert S. Langer, Newton, MA (US); Anna I. Mandinova, Newton, MA (US); Kristina Aleksandrova Todorova, Arlington, MA (US)

(21) Appl. No.: 17/293,507

(22) PCT Filed: Nov. 14, 2019
(86) PCT No.: PCT/US19/61478

§ 371 Date: May 13, 2021

Related U.S. Application Data
(60) Provisional application No. 62/767,764, filed on Nov. 15, 2018.

(51) Int. Cl. A61K 48/00; A61P 3/00

(52) U.S. Cl. CPC A61K 48/005; A61P 3/00

(57) ABSTRACT

A method for the systemic delivery of an enzyme to treat lysosomal storage disease of a subject is provided by creating genetically modified skin cells via topical introduction of a genetically engineered virus which delivers a nucleic acid encoding an enzyme or factor for expression by the skin cells, wherein the expressed enzyme or factor is secreted by the skin cells and is introduced into the circulatory system of the subject.

### RELATED APPLICATION DATA

This application claims priority to U.S. Provisional Application No. 62/767,764 filed on Nov. 15, 2018, which is hereby incorporated herein by reference in its entirety for all purposes.

### BACKGROUND

Gene therapy has shown great promise to prevent, treat and cure a variety of diseases and conditions in human and animals. The use of viruses to deliver nucleic acids to cells is generally known. Such viruses may be delivered by invasive methods requiring large doses of the virus. See Xiao, X., Li, J. & Samulski, R. J. Efficient long-term gene transfer into muscle tissue of immunocompetent mice by adeno-associated virus vector. J Virol 70, 8098-108 (1996). Such methods are challenging from a therapy or immunization perspective because of delivery efficiency of the nucleic acids to desired tissue in vivo. See Balazs, A. B., Ouyang, Y., Hong, C. M., Chen, J., Nguyen. S. M., Rao. D. S., An, D. S. & Baltimore, D. Vectored immunoprophylaxis protects humanized mice from mucosal HIV transmission. Nat Med 20, 296-300 (2014) and Brady, J. M., Baltimore, D. & Balazs, A. B. Antibody gene transfer with adeno-associated viral vectors as a method for HIV prevention. Immunol Rev 275, 324-333 (2017). One strategy for passive immunization uses the transcriptional machinery of host muscle cells. See Clark, K. R., Sferra. T. J. & Johnson, P. R. Recombinant adeno-associated viral vectors mediate long-term transgene expression in muscle. Hum Gene Ther 8, 659-69 (1997) and Kessler. P. D., Podsakoff, G. M., Chen. X., McQuiston, S. A., Colosi, P. C., Matelis, L. A., Kurtzman, G. J. & Byrne, B. J. Gene delivery to skeletal muscle results in sustained expression and systemic delivery of a therapeutic protein. Proc Natl Acad Sci USA 93, 14082-7 (1996). However, there is a continuing need in the art to improve the efficacy of gene therapy.

### SUMMARY

Aspects of the present disclosure are based on the use of genetically modified skin cells for the systemic delivery of an enzyme or factor to treat a subject deficient in the enzyme or factor, such as hemophilia A and B, and enzyme deficiencies including lysosomal storage diseases, hormone or factor deficiencies such as growth hormone deficiency, and other such deficiencies of an endogenous protein.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 is an illustration of one aspect of the present disclosure using sonic treatment to treat skin tissue before application of virus to the skin surface and delivery of the virus to skin cells where the skin cells express the foreign nucleic acid in the virus.

FIG. 2A depicts data directed to epidermal production of human α-galactosidase A (hGLA) as an exemplary enzyme to treat lysosomal storage disease using various viruses for delivery of the nucleic acid encoding hGLA using an EF1α promoter in the nucleic acid construct. Various viruses were effective at delivering the construct for expression.

FIG. 2B depicts data directed to full thickness skin production of human α-galactosidase A (hGLA) as an exemplary enzyme to treat lysosomal storage disease using various viruses for delivery of the nucleic acid encoding hGLA using an EF1α promoter in the nucleic acid construct. Various viruses were effective at delivering the construct for expression.

FIG. 2C depicts data showing production of human α-galactosidase A (hGLA) in response to a low dose and a high dose. Both high and low doses produced hGLA in the epidermis.

FIG. 3A depicts data of total human IgG produced in artificial epidermis using various viruses encoding bnAB (VRC01). Various viruses were effective at delivering the construct for expression.

FIG. 3B depicts data of total human IgG produced in full thickness artificial skin using various viruses encoding bnAB (VRC01). Various viruses were effective at delivering the construct for expression.

FIG. 4A depicts data of total human IgG produced (average production) in full thickness artificial skin maintained in a transwell culture using various viruses encoding bnAB (VRC01). Various viruses were effective at delivering the construct for expression.

FIG. 4B depicts data of total human IgG produced (total production) in full thickness artificial skin maintained in a transwell culture using various viruses encoding bnAB (VRC01). Various viruses were effective at delivering the construct for expression.

FIG. 5 depicts data plotting AAV tropism of secretion in epidermal and full thickness tissues.

FIG. 6A depicts data of bnAb production in response to escalated doses of virus in 3D-constructed tissue models.

FIG. 6B depicts dose response curves.

FIG. 6C depicts time response curves.

FIG. 7A depicts data of bnAb production using various viruses in human skin ex vivo.

FIG. 7B depicts data depicting luciferase expression in human skin ex vivo using different promoters.

FIG. 7C depicts images of luciferase expression in human skin ex vivo using different promoters.

FIG. 8 depicts data comparing administration of rAAV particles by intramuscular injection, intradermal injection and using ultrasound treatment as described herein before topical administration of virus. Ultrasound followed by topical administration provided gradually increasing amount of human IgG over 25 days.

### DETAILED DESCRIPTION

Embodiments of the present disclosure are directed to methods of treating enzyme or factor or protein deficiencies, such as congenital enzyme or factor or protein deficiencies, using gene therapy methods. For purposes of the present disclosure, enzyme or factor or protein deficiency is intended to describe an enzyme or factor or protein that is present in lower than normal amounts or an enzyme or factor or protein that is defective and does not carry out its intended function. Diseases associated with enzyme or factor or protein deficiencies are known to those of skill in the art and include lysosomal storage diseases. The enzymes or factors or proteins delivered by the methods described herein are considered therapeutic enzymes or factors or proteins to the extent that they are used as therapy to increase the amount of enzymes or factors or proteins in an individual at least to functioning levels. For ease of understanding, the terms enzyme, factor or protein can be used interchangeably for purposes of identifying a therapeutic agent to be delivered using the methods described herein.

Aspects of the present disclosure are directed to delivering nucleic acid molecules of interest encoding one or more therapeutic enzymes or factors or proteins via recombinant viruses to a skin tissue in order to treat an enzyme or factor or protein deficiency. The present disclosure describes a method of systemic delivery of one or more therapeutic enzymes or factors or proteins to a subject including genetically modifying target skin cells within skin of a subject using an engineered virus.

The engineered virus includes one or more viral genomic nucleic acid sequences and one or more foreign nucleic acid sequences encoding one or more enzymes or factors or proteins. The one or more viral genomic nucleic acid sequences and the one or more foreign nucleic acid sequences encoding one or more enzymes or factors or proteins are introduced into the target skin cells to produce genetically modified target skin cells. The genetically modified target skin cells produce the one or more therapeutic enzymes or factors or proteins. The one or more therapeutic enzymes or factors or proteins are excreted from the genetically modified skin cells and is introduced systemically within the subject via the bloodstream.

According to one aspect, the genetically modified target skin cells may contain the genetic elements to also produce the engineered virus which replicates intradermally between target cells. In this manner, engineered virus carrying the one or more foreign nucleic acid sequences encoding one or more therapeutic enzymes or factors or proteins is transmitted in vivo between target skin cells to create additional genetically modified skin cells producing the one or more therapeutic enzymes or factors or proteins. The one or more therapeutic enzymes or factors or proteins is excreted from the genetically modified skin cells and is introduced systemically within the subject via the bloodstream.

According to one aspect, an engineered virus is administered to the skin of the subject in a manner to direct the engineered virus to the target skin cells. Various administration methods are contemplated including topical application to skin and other methods known to those of skill in the art and as described herein.

According to one aspect, the skin of the subject may be treated so as to permeabilize the stratum corneum of the skin to the presence of the engineered virus or otherwise improve efficiency of the engineered virus to traverse the stratum corneum to the target skin cells. After treating the skin surface, the engineered virus may be administered to the skin surface, such as by topical administration, and the engineered virus may be directed to or passively diffuse to the target skin cells whereupon the engineered virus infects the target cells to include the one or more nucleic acid sequences encoding one or more therapeutic enzymes or factors or proteins.

Accordingly, in exemplary embodiments, methods described herein include two major steps. In step one, ultrasound or other methods are applied to a skin tissue to increase tissue permeation. In step two, recombinant viruses carrying foreign nucleic acid molecule(s)/gene(s) of interests are delivered to the skin cells. The virus replicates to other cells within a target cell population using a viral replication mechanism so as to intradermally provide target cells with one or more nucleic acid sequences encoding one or more therapeutic enzymes or factors or proteins. The one or more therapeutic enzymes or factors or proteins are produced by the genetically modified target cells and the one or more therapeutic enzymes or factors or proteins are excreted from the genetically modified target cells and into the blood stream of the subject, so as to provide a systemic administration of the one or more therapeutic enzymes or factors or proteins.

According to one aspect, the one or more therapeutic enzymes or factors or proteins are excreted from the genetically modified target cells in a manner to provide a prolonged release of the one or more therapeutic enzymes or factors or proteins into the bloodstream of the subject. Embodiments of the present disclosure are directed to a method of delivering a recombinant virus including a foreign nucleic acid encoding an enzyme, factor or protein to a skin tissue including applying ultrasound to the skin tissue, and administering the recombinant virus to the skin tissue. According to one aspect, the recombinant virus is delivered to the skin tissue of a subject in vivo.

According to one aspect, a delivery platform is provided that utilizes a subject's skin, such as mammalian skin, to enable a single-step, extended production (such as year-long production) of one or more therapeutic enzymes or factors or proteins wherein enzyme or factor or protein-encoded vectors are topically administered to skin in a non-invasive manner so as to treat or prevent a lysosomal storage disease. Skin cells are provided with non-integrative viral vectors which, according to one embodiment, may lack specific cytotoxicity and pathogenicity. According to one aspect, delivery of the viral vectors is achieved by noninvasive or “needleless” methods. Such noninvasive or “needleless” methods may also include breakage of the stratum corneum using methods described herein or which become apparent based on the present disclosure. The protective skin layer known as the stratum corneum is disrupted so as to provide entry sites through the stratum corneum to cells below the stratum corneum. The cells are to be genetically modified by viral infection. The genetic modification of skin cells to include the enzyme or factor or protein-encoded vectors provides for long-lived and efficient translation of the therapeutic enzyme or factor or protein in vivo to provide a safe and effective treatment of enzyme deficiencies, such as those associated with lysosomal storage diseases.

According to one aspect, skin is pretreated using noninvasive technology, such as ultrasound or microdermabrasion, to permeabilize or score or remove the stratum corneum. The engineered virus, such as an enzyme or factor or protein-encoding adeno-associated virus (“AAV particles”) is administered to the skin or otherwise delivered to the skin, which may be a section of skin near active lymph nodes. According to one aspect, target skin cells (such as dermal fibroblasts) endosome the AAV particles and the AAV particles release the DNA contained therein into the skin cell nucleus. The skin cells translate and secrete the one or more enzymes or factors or proteins to the blood stream. The enzymes or factors or proteins are present within the blood system for therapy or prevention. In this manner, the skin may be transformed into an in vivo bioreactor for the production of therapeutic enzymes or factors or proteins for transfer into the blood stream, for example to treat enzyme deficiencies, such as those associated with lysosomal storage diseases.

### Lysosomal Storm Diseases

Congenital enzyme deficiencies are genetic metabolic diseases characterized by enzyme deficiencies that affect various parts of the body including brain, central nervous system, heart, skeleton, skin. There are more than 50 diseases described as lysosomal storage diseases including Fabry. Gaucher's, Hunter, Tay Sach's, Batten, Pompe, Mucolipidosis, Niemann-Pick, Krabbe, and Hurler. Lysosomal storage diseases or deficiencies are characterized by a deficiency of an enzyme required for the metabolism of large molecules such as lipids, glycoproteins (sugar-containing proteins), or mucopolysaccharides. These lysosomal enzyme deficiencies lead to abnormal build-up of such large molecules or toxins in cells and interfere with lysosomes' normal function, and can lead to cell death. Lysosomal enzymes will become apparent to those of skill in the art based on the present disclosure. The signs and symptoms of lysosomal storage disorders manifest over time and are progressive by nature. Most of these disorders are inherited in an autosomal recessive manner with a few exceptions such as Fabry disease and Hunter syndrome which are X-linked recessive. Aspects of the present disclosure are directed to the identification of one or more deficient enzymes, such as deficient enzymes associated with lysosomal storage disorders. The one or more enzymes are delivered into the blood stream by being expressed within skin cells, such as by the methods described herein.

Exemplary enzymes, factors or proteins that when are deficient are associated with lysosomal storage disorders include α-galactosidase A (GLA), α-galactosidase B, β-galactosidase (GLB1), neuraminidase 1 (NEU1), glucocerebrosidase, ceramidase (ASAH1), beta-hexosaminidase, hexosaminidase A, hexosaminidase B, sphingomyelinase, sulphatase, galactocerebrosidase, lysosomal acid lipase (LAL), glucocerebrosidase, arylsulfatase A (ARSA), arylsulfatase B (ARSB), formylglycine-generating enzyme (FGE), α-L-iduronidase, iduronidase, iduronate sulfatase, iduronate-2-sulfatase (I2S), heparan sulfamidase, n-acetylglucosaminidase, heparan-α-glucosaminide, N-acetyltransferase, acetyltransferase, N-acetylglucosamine-6-sulfatase, galactose-6-sulfate sulfatase, N-acetylgalactosamine-4-sulfatase, galactosamine-6-sulfate sulfatase, β-glucuronidase, hyaluronidase, HYAL1, HYAL2, HYAL3, HYAL4, HYAL5, SPAM1, PH-20, HYAL6, HYALP1, hyaluronoglucosidase, hyauronoglucuronidase, cathepsin A, glycosidase, α-N-acetyl neuraminidase (sialidase), phosphotransferase, mucolipid1, palmitoyl-protein thioesterase, tripeptidyl peptidase, PPT1, TPP1, α-D-mannosidase, beta-mannosidase, aspartylglucosaminidase, alpha-L-fucosidase, alpha-glucosidase, cystinosin, cathepsin K, sialin, solute carrier family 17, or prosaposin. One of skill in the art will be able to identify other enzymes associated with lysosomal storage disorders based on the present disclosure.

It is to be understood that one of skill in the art would understand that aspects of the present disclosure include nucleic acid sequences encoding the enzymes or factors and that such nucleic acid or gene sequences can be readily identified by one of skill in the art. It is to be understood that the present disclosure contemplates using the known nucleic acid or gene sequence or a nucleic acid sequence having at least 70%, 80%, 85%, 90%, 95%, 96%, 97%, 98%, or 99% homology thereto. One of skill would understand based on the present disclosure that one can use the enzyme or factor or a modified or mutant enzyme or factor that retains the enzyme or factor activity.

### Subjects and Target Cells

According to one aspect, the methods are carried out on a subject which may be a human or non-human mammal. The non-human mammal may be a mouse, rat, cow, pig, sheep, goat, horse, dog or cat.

According to one aspect, the methods are carried out on skin as described herein and the vectors or viral vectors are transmitted to skin cells as described herein as target skin cells. Different skin layers, structures and cells can be targeted for nucleic acid or gene delivery according to certain embodiments of the disclosed methods. The skin is composed of diverse cells derived from three distinct embryonic origins: neurectoderm, mesoderm, and neural crest. Recombinant viral vectors can be delivered to one or more of the three layers of the skin: the epidermis, dermis, and hypodermis. The epidermis, the outermost layer, is primarily composed of stratified squamous epithelium of keratinocytes, which is derived from neurectoderm and comprises over ninety percent of epidermal cells. The stratified squamous epithelium is further divided into four layers, starting with the outermost layer: stratum corneum (SC), stratum granulosum (SG), stratum spinosum (SS), and stratum basale (SB). Cells of the epidermis including keratinocytes which are responsible for the cohesion of the epidermal structure and the barrier function, pigment-containing melanocytes, antigen-processing Langerhans cells, and pressure-sensing Merkel cells can be targeted by the viral vectors.

The dermis is a connective tissue that is responsible for the mechanical properties of the skin. It is composed of fibroblasts of mesoderm origin, which lie within an extracellular specialized matrix. Collagens are interwoven with elastin, proteoglycans, fibronectin, and other components. The epidermis and dermis are connected by a basement membrane that is composed of various integrins, laminins, collagens, and other proteins that play important roles in regulating epithelial-mesenchymal cross-talk. The superficial papillary dermis is arranged in ridge-like structures called the dermal papillae, which contains microvascular and neural networks and extends the surface area for these epithelial-mesenchymal interactions. Sebaceous glands, eccrine glands, apocrine glands and hair follicles are of neurectoderm origin and develop as downgrowths of the epidermis into the dermis. Outer root sheath of the hair follicle is contiguous with the basal epidermal layer. In addition, the dermis also contains blood vessels and lymphatic vessels of mesoderm origin, and sensory nerve endings of neural crest origin. The hypodermis, which is deep to the dermis, is composed primarily of adipose tissue of mesoderm origin, and separates the dermis from the underlying muscular fascia. Vectors and viral vectors can also target these cells, glands, and structures of the dermis and hypodermis as described above.

Recombinant viral vectors can also target skin-specific stem cells which possess the ability for skin tissue to self-renew. Multipotent or unipotent skin stem cells are slowly-cycling cells that reside in at least five distinct niches in the skin: basal (innermost) layer of epidermis, hair follicle bulge, base of sebaceous gland, dermal papillae, and dermis. Not only are these stem cells critical for the long-term maintenance of the skin tissue but also are activated by wounding to proliferate and regenerate the tissue. Skin specific resident T cells are also target skin cells within the present disclosure. Skin-specific stem cells include hair follicle stem cells for hair follicle and continual hair regeneration, melanocyte stem cells giving rise to the melanocytes in both the hair matrix and epidermis, stem cells at the base of the sebaceous gland for continually generating terminally differentiated sebocytes, which degenerate to release lipids and sebum through the hair canal and lubricate the skin surface, mesenchymal stem cells that giving rise to fibroblasts, nerves and adipocytes, and a skin-derived precursor stem cell (SKP) distinct from mesenchymal stem cells.

It is to be understood that the basic concepts of the present disclosure described herein are not limited by cell type. For example, target skin cells include cell types around hair follicles as the method may be applied to haired regions for delivery as the vectors or viral vectors may more easily penetrate through such skin areas. It is to be understood that more than one cell type can be targeted at the same time by using a mixture of hybrid AAVs directed to each cell type in a plurality of cell types, such as to be administered in one cocktail formulation where it is desired to enhance efficiency of infectivity and achieve broad tropism.

According to one aspect, the target cells described herein may be skin cells. According to one aspect, the skin cells are in vivo, in vitro or ex vivo. Exemplary target skin cells are dermal fibroblasts. According to one aspect, the skin cells are mammalian skin cells. Mammals include, but are not limited to murines, simians, humans, farm animals, sport animals, and pets. Tissues, cells and their progeny of a biological entity obtained in vivo or cultured in vitro are also encompassed. According to one aspect, the skin cells are human skin cells. According to one aspect, the target cells are present in sufficient number so as to produce a sufficient amount of the therapeutic enzyme, factor or protein to provide a sufficient concentration of the therapeutic enzyme, factor or protein within the blood of a subject so as to provide therapeutic treatment.

Dermal fibroblasts account for a total of 2.6×10¹⁰ cells at an average surface density of 1.3×10⁶ cells/cm². Dermal fibroblasts are relatively transcriptionally uncommitted and they have long cell cycle of about 54-60 days. Dermal fibroblasts produce monoclonal antibodies upon retroviral gene transfer in vitro (see Noel, D., Pelegrin, M., Brockly, F., Lund, A. H. & Piechaczyk, M. Sustained systemic delivery of monoclonal antibodies by genetically modified skin fibroblasts. J Invest Dermatol 115, 740-5 (2000) hereby incorporated by reference in its entirety) and in skin grafts in immunocompetent mice (see Noel, D., Dazard. J. E., Pelegrin, M., Jacquet, C. & Piechaczyk, M. Skin as a potential organ for ectopic monoclonal antibody production. J Invest Dermatol 118, 288-94 (2002) hereby incorporated by reference in its entirety). Exemplary target skin cells are present in a sufficient amount, are relatively transcriptionally uncommitted and have long cell cycle. Target cells can also include any skin cell having the characteristics described above, such as epidermal progenitors. See Khavari, P. A., Rollman, O. & Vahlquist, A. Cutaneous gene transfer for skin and systemic diseases. J Intern Med 252, 1-10 (2002) hereby incorporated by reference in its entirety.

According to one aspect, a skin surface area and skin location for administration of engineered viruses to result in a sufficient production of a therapeutic enzyme, factor or protein is determined. For calculations of fibroblast translational capacity and necessary surface area of transduction, estimations for cell densities in the two dermal layers: papillary dermis, occupying ˜10% of the total dermal thickness, and reticular dermis—the rest, 90% are used. See Sender, R., Fuchs, S. & Milo, R. Revised Estimates for the Number of Human and Bacteria Cells in the Body. PLoS Biol 14, e1002533 (2016) hereby incorporated by reference in its entirety. Due to variations in cell density as a function of dermal depth, the cell surface density is two orders larger in the papillary dermis versus that in the reticular dermis. An exemplary surface skin area for the transduction of 10⁸ cells (previously reported to output 10 μg/mL in mouse serum) at 50% efficiency is about 142 cm², or a patch of about 12 cm by 12 cm in a 100-kilogram individual. It is to be understood that the estimates of cell number to provide a desired output of therapeutic enzyme, factor or protein may be based on empirical observations of genetically modified fibroblasts embedded in artificial matrices before implantation in vivo. Therefore, estimates are not exact predictions but rather, useful, though rough order-of-magnitude estimates of an exemplary upper bound constraint for cell number and area requirement. One of skill will readily be able to determine suitable surface areas for delivery of various concentrations of therapeutic enzyme, factor or protein into the circulatory system or other system suitable for systemic administration of therapeutic enzyme, factor or protein, such as for the treatment of enzyme deficiency associated with lysosome storage diseases. Such therapeutic enzymes useful in the methods described herein are readily identifiable based on the present disclosure.

According to the present disclosure, secretion capacity may be assessed ex vivo. Production efficiency is tested in human skin explants taken from patients' anatomical sites characterized with thick dermis and thin epidermis at doses and surface areas necessary to achieve ˜10-100 μg/mL (an optimal concentration to translate to ˜1 μg/mL in human in vivo). Dermal-epidermal ratios (DERs) are high for anterior abdomen, forehead, anterior chest, and thigh in human skin. According to one aspect, an exemplary anatomical site for engineered virus administration is near the small pelvis, is highly vascularized and is in close proximity to active lymphatics. An exemplary anatomical site is anterior abdomen (with a score of DER=8.1), or thigh (of DER=5.7). According to one aspect, dermal fibroblasts in living skin of the anterior abdomen are targeted with non-integrative viral vectors encoding therapeutic enzyme, factor or protein with high efficiency and long temporal secretion to the blood stream.

### Plasmids, Vectors and Viral Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” or “operatively linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). Vectors according to the present disclosure include those known in the art as being useful in delivering genetic material into a cell and would include regulators, promoters, enhancers, nuclear localization signals (NIS), start codons, stop codons, a transgene etc., and any other genetic elements useful for integration and expression, as are known to those of skill in the art.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the one or more foreign nucleic acids to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. However, it is to be understood that useful viral vectors may also include the genetic sequences for replication and the capsid, when it is desired that the virus be replicated and transmitted from cell to cell. In such a method, the virus replicates and transmits the one or more foreign nucleic acid sequences from cell to cell for expression.

According to one aspect, methods described herein may use a viral plasmid without the capsid. Such a viral plasmid is referred to in the art as a naked viral plasmid. That is, the viral plasmid will have the ITR regions and all nucleic acid sequence elements required for transcription but delivered naked without the capsid.

According to one aspect, viral vectors may be selected based on the ability to target cell types in a specific manner. According to one aspect, exemplary viruses may be identified based on the parameters described herein. The use of recombinant RNA or DNA viral based vector systems for the delivery of nucleic acids takes advantage of highly evolved processes for targeting a virus to specific cells in the skin tissue and trafficking the viral payload to the nucleus. According to certain embodiments, recombinant viral vectors can be administered directly to the skin of a subject (in vivo) or they can be administered to skin tissues or cells in vitro, and skin tissues or cells that were modified by the recombinant viruses may optionally be grafted or administered back to the subject (ex vivo).

Conventional recombinant or engineered viral based vector systems can include retroviral, lentivirus, adenoviral, adeno-associated virus (AAV), vaccinia virus and herpes simplex virus vectors for gene transfer. Of these viral vectors, recombinant AAV is thought to be the safest due to its lack of pathogenicity. According to one aspect, the engineered virus is a recombinant AAV of serotype 1, 2, 3, 4, 5, 6, 7, 8 or 9. According to one aspect, exemplary viruses include recombinant AAVs of serotype AAV1, AAV2, AAV5, AAV6.2, AAV7, AAV8, AAV9, AAVDJ, AAV10, AAVhu11, AAVrh32.22, AAV-Anc80, or AAV-Anc113. Integration in the host genome is possible with the retrovirus, lentivirus, and adeno-associated virus gene transfer methods, often resulting in long term expression of the inserted transgene encoding the therapeutic enzyme, factor or protein. Additionally, high transduction efficiencies using these recombinant viruses have been observed in many different cell types and target tissues.

In certain embodiments, following ultrasound treatment of the skin, rAAV vectors containing genes of interest are topically applied to the skin tissue and let passively diffuse to reach skin cells in both epidermal and dermal skin layers. The tropism of an AAV can be altered by different capsid proteins. A person skilled in the art can select appropriate rAAV serotype, including serotypes 1-9 based on the tropism for a particular cell type.

According to one aspect, rAAV vectors or particles are utilized which lack the capability to replicate, i.e., they are nonreplicating. Such AAV vectors are known to those of skill in the art for delivering a payload nucleic acid sequence encoding therapeutic enzyme, factor or protein. Such vectors or particles are delivered to a cell. For example, one or more AAV particles are delivered per cell, where an exemplary infectivity ratio is typically 100:1 virus to cell, and is dose-dependent. Once infected by the virus, the cell has machinery to transcribe the viral DNA (which is circular double stranded DNA) encoding for a therapeutic enzyme, factor or protein known to those of skill in the art and which may be referred to as a payload. The infected cell has the cellular machinery to properly make the therapeutic enzyme, factor or protein which is then secreted to the blood stream and then the lymphatics.

According to one aspect, a replicating virus may be used in gene therapy methods described herein. Such an approach utilizes a molecular switch for activating and/or deactivating the replication capability of the virus. Both nonreplicating and replicating viruses are used as treatment or prophylaxis for various conditions, ranging from immunotherapy in cancer, autoimmune diseases whose treatment require monoclonal antibodies, to infectious diseases where passive production of antibodies enables immunity and are especially useful for delivering one or more foreign nucleic acids encoding therapeutic enzyme, factor or protein as described herein.

Exemplary viral vectors may be identified by multiplexed screening of hybrid capsid variations of adeno-associated viruses (“AAVs”). Hybrid AAV constructs typically exhibit less immunogenicity than the wild-type AAV, and have greater tissue specificity.

A large set of existing viral serotypes is optimized, synthesized and tested in human organotypic cultures. Human abdominal skin is cultured ex vivo, using native fluorescence of reporter genes, FACS, and in situ screening approaches. The method is high-throughput, allows for combinatorial optimization, and accounts for donor-to-donor variability related to immune response and metabolic state. According to one aspect, a human skin explant model is utilized that preserves the physiological complexity, the proliferative capacity and the structural integrity of all skin components for up to 28 days. See Frade, M. A., Andrade, T. A., Aguiar, A. F., Guedes, F. A., Leite, M. N., Passos, W. R., Coelho, E. B. & Das, P. K. Prolonged viability of human organotypic skin explant in culture method (hOSEC). An Bras Dermatol 90, 347-50 (2015); Manevski, N., Swart, P., Balavenkatraman, K. K., Bertschi. B., Camenisch, G., Kretz, O., Schiller, H., Walles, M., Ling, B., Wettstein, R., Schaefer, D. J., Itin, P., Ashton-Chess, J., Pognan, F., Wolf, A. & Litherland, K. Phase II metabolism in human skin: skin explants show full coverage for glucuronidation, sulfation, N-acetylation, catechol methylation, and glutathione conjugation. Drug Metab Dispos 43, 126-39 (2015); and Xu. W., Jong Hong. S., Jia, S., Zhao, Y., Galiano. R. D. & Mustoe, T. A. Application of a partial-thickness human ex vivo skin culture model in cutaneous wound healing study. Lab Invest 92, 584-99 (2012) each of which are hereby incorporated by reference in its entirety.

Viable explants are utilized with a surface area of 15-20 mm to enable topical treatment with test agents and compositions. See Kolev, V., Mandinova, A., Guinea-Viniegra. J., Hu, B., Lefort, K., Lambertini, C., Neel. V., Dummer, R., Wagner, E. F. & Dotto, G. P. EGFR signalling as a negative regulator of Notch1 gene transcription and function in proliferating keratinocytes and cancer. Nat Cell Biol 10, 902-11 (2008) and Neel, V. A., Todorova, K., Wang, J., Kwon, E., Kang, M., Liu, Q., Gray, N., Lee, S. W. & Mandinova, A. Sustained Akt Activity Is Required to Maintain Cell Viability in Seborrheic Keratosis, a Benign Epithelial Tumor. J Invest Dermatol 136, 696-705 (2016) each of which are hereby incorporated by reference in its entirety.

According to one aspect, rAAV vector serotypes exhibit tissue specificity and efficiency of gene transfer which can be determined by methods known in the art. For example, the human explant model is used to determine and optimize the efficiency of AAV-based delivery of therapeutic enzymes, factors or proteins to certain cellular components of the skin, the dose response and the temporal dynamics of secretion of therapeutic enzymes, factors or proteins to the surrounding medium. The cellular tropism of a pool of AAV serotypes is tested. Exemplary candidates are selected with high degree of specificity to dermal fibroblasts, and efficacy of transcription and translation of therapeutic enzymes, factors or proteins in human dermal fibroblasts is determined. A high-throughput approach is used to analyze a large pool of explants (maintained in multi-well organotypic chambers) using multiple infection doses and serotypes while accounting for donor-to-donor variability.

Keratinocytes, epidermal stem cells, hair follicle stem cells, sebocytes, dermal fibroblasts, adipocytes precursors, mesenchymal stem cells and endothelial cells are determined as preferential targets of the AAVs. Exemplary target cells are dermal fibroblasts due to their less differentiated state, uncommitted and potent transcriptional and translational machinery and fairly high abundance in the entire superficial dermis. See Krueger, G. G. Fibroblasts and dermal gene therapy: a minireview. Hum Gene Ther 11, 2289-96 (2000) and Birchall, J., Coulman, S., Pearton, M., Allender, C., Brain, K., Anstey, A., Gateley, C., Wilke, N. & Morrissey, A. Cutaneous DNA delivery and gene expression in ex vivo human skin explants via wet-etch micro-fabricated micro-needles. J Drug Target 13, 415-21 (2005) each of which are hereby incorporated by reference in its entirety. Other cell types may also be used such as proliferating keratinocytes, particularly of those residing at the basal layer.

According to one aspect, different AAV serotypes can be selected to provide optimal AAV infection dose for maximum expression of the therapeutic enzymes, factors or proteins. According to one aspect, efficacy of secretion is evaluated in human skin explants and the ability of the infected cells inside the intact tissue to produce and secrete the respective therapeutic enzymes, factors or proteins. According to one aspect, the explant culture system positions the dermal (bottom) surface of the tissue on a Biopore™ (PTFE) membrane cell strainer with the epidermis (the top) facing up. The dermis is kept in constant contact with the growth medium while the epidermis is exposed to air. This allows for a proper proliferation and differentiation of the keratinocytes (as in vivo) and mimics a contact of the dermis with the circulatory system. The therapeutic enzymes, factors or proteins produced by the infected skin cells are analyzed by standard protocols such as ELISA of the conditioned medium.

### Nucleic Acid Constructs

According to one aspect, nucleic acid constructs are provided for transmission into skin cells of a subject. The nucleic acid constructs may be included within a virus for introduction into a cell and for expression by the cell. The nucleic acid construct encoding the therapeutic enzyme, factor or protein may be referred to as a payload construct. The payload constructs are expressed by the cell into which they are introduced by the plasmids, vector or viral vectors in which they are included. One of skill will be able to identify suitable plasmids, vectors and viral vectors and will also be able to design suitable nucleic acid constructs including one or more payload nucleic acids for expression by a cell.

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. Regulatory elements may also direct expression in an inducible manner, such as in a small-molecule dependent or light-dependent manner. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter and Pol II promoters described herein. Exemplary promoters include CMV, CAG, UBC, EF1α, CASI and CASI-WRPE.

Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, antibodies, nanobodies, or peptides, including fusion proteins or peptides, encoded by nucleic acids as described herein.

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

Exemplary nucleic acid constructs for the payload nucleic acid construct or the one or more foreign nucleic acid sequences may include the regulatory elements within a backbone sequence as is known in the art for expressing the payload nucleic acid.

### Skin Treatment and Cutaneous Foreign Nucleic Acid Transfer

According to one aspect, one or more foreign nucleic acids encoding therapeutic enzymes, factors or proteins within a virus are transmitted to skin cells for a gene-based systemic protein delivery method as described herein. The skin is an exemplary organ or tissue for system delivery of a therapeutic or prophylactic agent because of its accessibility, rich vascularization and ability to release skin-produced polypeptides (such as engineered antibodies) to the blood stream. See Khavari, P. A., Rollman, O. & Vahlquist, A. Cutaneous gene transfer for skin and systemic diseases. J Intern Med 252, 1-10 (2002); Birchall, J., Coulman, S., Pearton, M., Allender, C., Brain, K., Anstey, A., Gateley. C., Wilke. N. & Morrissey. A. Cutaneous DNA delivery and gene expression in ex vivo human skin explants via wet-etch micro-fabricated micro-needles. J Drug Target 13, 415-21 (2005) and Coulman, S. A., Barrow, D., Anstey, A., Gateley, C., Morrissey, A., Wilke, N., Allender, C., Brain. K. & Birchall, J. C. Minimally invasive cutaneous delivery of macromolecules and plasmid DNA via microneedles. Curr Drug Deilv 3, 65-75 (2006) each of which is hereby incorporated by reference in its entirety. Skin based delivery methods also improves patient compliance together with precisely controlled and if desired pulsatile delivery of the polypeptide as a therapeutic or prophylactic agent agent.

As the skin provides a primary barrier to microbial invasion and desiccation, cutaneous tissue, however, possesses substantial obstacles to effective insertion of foreign DNA and/or viral particles. While various technologies are established for transdermal delivery of small and large molecules (see Guy, R. H., Hadgraft, J. & Bucks, D. A. Transdermal drug delivery and cutaneous metabolism. Xenobiotica 17, 325-43 (1987); Prausnitz, M. R. & Langer, R. Transdermal drug delivery. Nat Biotechnol 26, 1261-8 (2008); and Prausnitz, M. R., Mitragotri, S. & Langer, R. Current status and future potential of transdermal drug delivery. Nat Revs Drug Discos' 3, 115-24 (2004)), methods described herein are directed to the skin cells being genetically modified by transduction with naked or viral vectors, such as rAAVs, as foreign nucleic acid carriers so as to provide intradermal delivery. Methods described herein are well controlled and highly efficient in therapeutic enzyme, factor or protein production intradermally with minimal irritation, no wound healing and regenerative reaction to sustain prolonged expression of the therapeutic enzymes, factors or proteins.

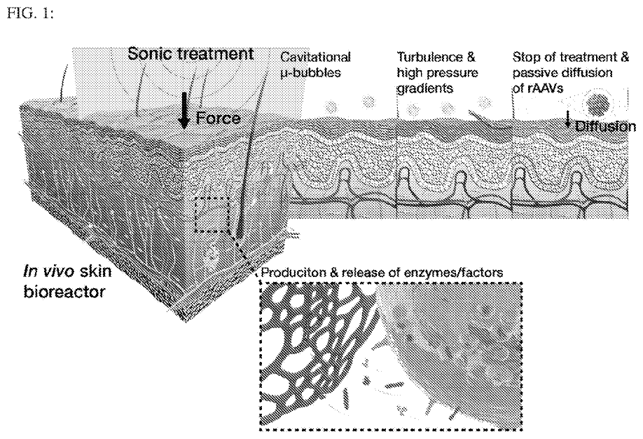

According to one aspect, the skin is treated to facilitate or enable virus (AAV-vectored antibody) penetration through the stratum corneum and into the epidermal and dermal layers, and associated skin cells, below. The present disclosure provides a two-step gene transfer to skin that does not require manipulation ex vivo. As shown in the schematic in FIG. 1, in the first step, a patch of skin is treated with cavitational ultrasound. A formulation of viral particles is then topically administered. This approach utilizes in situ mechanical disruption of the stratum corneum, the single topmost protective cell layer of the skin, along with the natural ability of viral vectors to deliver genetic material to cells. To achieve a precise and yet minimally invasive delivery, this gene transfer modality combines two steps: 1) a uniform needleless tissue permeabilization and intradermal delivery harnessing forces generated by cavitational pressure through low frequency ultrasonic waves; 2) topical viral passive delivery facilitating precision in cell type transduction and targeted delivery of transgenes to main components of the skin for optimized secretion.

According to one aspect, ultrasound is used to treat the skin prior to application of the virus to the skin to increase skin tissue permeation. An exemplary method to treat the skin is cavitational, low-frequency ultrasound applied in a manner to reversibly disrupt the cutaneous stratum corneum and to enable rAAV transport into the epidermis, the papillary and reticulous dermis avoiding injury of the surrounding tissues. A person skilled in the art can choose the appropriate ultrasound device according to an application. A person skilled in the art can determine the frequency, intensity and duration of ultrasound application that is effective for a specific purpose. The ultrasonic pre-treatment of skin tissue improves tissue diffusivity by increasing its effective diffusion coefficient. This process is enabled by the disruption of the skin's stratum corneum.

Cavitational ultrasound has been applied successfully in vivo in animals for the delivery of chemical compounds and RNA to the gastrointestinal tract. See Schoellhammer, C. M., Lauwers, G. Y., Cmettel, J. A., Oberli, M. A., Cleveland, C., Park, J. Y., Minahan, D., Chen, Y., Anderson, D. G., Jaklenec, A., Snapper, S. B., Langer, R. & Traverso, G. Ultrasound-Mediated Delivery of RNA to Colonic Mucosa of Live Mice. Gastroenterology 152, 1151-1160 (2017) and Schoellhammer, C. M., Schroeder, A., Maa, R., Lauwers, G. Y., Swiston, A., Zervas, M., Barman, R., DiCiccio, A. M., Brugge, W. R., Anderson, D. G., Blankschtein, D., Langer, R. & Traverso, G. Ultrasound-mediated gastrointestinal drug delivery. Sci Transl Med 7, 310ra168 (2015) each of which are hereby incorporated by reference in its entirety. Notably, this technology has already been approved by the FDA for enhanced lidocaine delivery through the skin. See Becker, B. M., Helfrich, S., Baker, E., Lovgren, K., Minugh, P. A. & Machan. J. T. Ultrasound with topical anesthetic rapidly decreases pain of intravenous cannulation. Acad Emerg Med 12, 289-95 (2005) and Skarbek-Borowska, S., Becker, B. M., Lovgren, K., Bates, A. & Minugh, P. A. Brief focal ultrasound with topical anesthetic decreases the pain of intravenous placement in children. Pediatr Emerg Care 22, 339-45 (2006) each of which is hereby incorporated by reference in its entirety. Cavitational ultrasound uses low frequency (<100 kHz) to form, oscillate and collapse bubbles in an ultrasonic pressure field between the ultrasound probe and the skin surface. See Ogura, M., Paliwal, S. & Mitragotri, S. Low-frequency sonophoresis: current status and future prospects. Adv Drug Deliv Rev 60, 1218-23 (2008) and Paliwal, S., Menon, G. K. & Mitragotri, S. Low-frequency sonophoresis: ultrastructural basis for stratum corneum permeability assessed using quantum dots. J Invest Dermatol 126, 1095-101 (2006) each of which is hereby incorporated by reference in its entirety. According to one aspect, cavitational ultrasound is used to facilitate the transient permeabilization of the stratum corneum and to propel the viral particles inside the skin without damaging deeper tissues. Cavitational ultrasound is used without morphological signs of irritation, induced wound healing or compensatory regenerative response in the epidermis or underlying dermis both ex vivo and in vivo. According to one aspect, rAAV penetration into the skin is facilitated by treatment with cavitational ultrasound at 20 kHz, which is applied at an intensity of less than 8 W/cm² for up to one minute at a 50% duty cycle, for example, using a hand-held ultrasound device.

In some embodiments, the ultrasound is applied at a frequency between about 10 kHz and about 100 kHz, about 10 kHz and about 20 kHz, about 10 kHz and about 50 kHz such as at a frequency of about 10 kHz, 20 kHz, 30 kHz, 40 kHz, 50 kHz, 60 kHz, 70 kHz, 80 kHz, 90 kHz and 100 kHz. In other embodiments, the ultrasound is applied at an intensity between about 1 W/cm² and about 300 W/cm², about 1 W/cm² and about 10 W/cm², about 10 W/cm² and about 300 W/cm², about 100 W/cm² and about 300 W/cm², about 200 W/cm² and about 300 W/cm², such as about 1, 2, 3, 4, 5, 6, 7, 8, 9 and 10 W/cm². In some embodiments, the ultrasound is applied for a duration between about one minute to about 10 minutes such as for a duration of about 1, 2, 3, 4, 5, 6, 7, 8, 9 and 10 minutes. In some embodiments, the ultrasound is applied at duty cycles in the range between about 10% and 100%, or about 20% and 100%. In some embodiments, the ultrasound is applied at duty cycles in the range between about 10%, 25%, 50%, 75% and 100%. In certain embodiments, the ultrasound is applied topically or intra-dermally.

According to one aspect, microdermabrasion is used to treat the skin prior to application of the virus to the skin. Microdermabrasion is an FDA approved process first introduced in the early nineties but quickly gained popularity for the treatment of scars, acne and other skin conditions due to high efficacy and simplicity of application. See Andrews, S., Lee, J. W. & Prausnitz. M. Recovery of skin barrier after stratum corneum removal by microdermabrasion. AAPS PharmSciTech 12, 1393-400 (2011); Andrews, S. N., Zarnitsyn, V., Bondy. B. & Prausnitz, M. R. Optimization of microdermabrasion for controlled removal of stratum corneum. Int J Pharm 407, 95-104 (2011) and Gill, H. S., Andrews, S. N., Sakthivel, S. K., Fedanov, A., Williams, I. R., Garber, D. A., Priddy, F. H., Yellin, S., Feinberg, M. B., Staprans, S. I. & Prausnitz, M. R. Selective removal of stratum corneum by microdermabrasion to increase skin permeability. Eur J Pharm Sci 38, 95-103 (2009) each of which is hereby incorporated by reference in its entirety. Mechanistically, microdermabrasion involves impingement of micro-particles on the skin, which are then removed under vacuum along with the abraded dead superficial skin layer. In this manner, the stratum corneum is removed in a controlled fashion, without any collateral damage to the underlying viable cells of the skin.

### Vector Design

According to one aspect, enhanced versions of viral cis vectors are designed or engineered to provide high transcription efficiency and capacity to translate encoding sequences of interest. One of skill may identify suitable vector designs through sequence optimizing practices used for the expression of single and double cistronic vectors and then testing in a highly homogenous reproducible in vitro system utilizing primary human cell cultures.

According to one aspect, methods are provided for the sustained production and cell-specific transfer of therapeutic enzymes, factors or proteins to skin cells. Methods described herein are designed to limit off-target cellular tropism to fast proliferating epidermal cells which can lead to rapid clearance of transgenes. According to one aspect, skin cells such as fibroblasts or keratinocytes are genetically engineered for production of a therapeutic enzyme, factor or protein. Such cells produce therapeutic enzymes, factors or proteins. Fibroblasts are known to produce monoclonal antibodies, though using cell expansion from skin biopsies, ex vivo genetic modification with retroviral vectors, and intraperitoneal implantation of genetically modified cells embedded in artificial collagen matrix in mice. See Noel, D., Pelegrin, M., Brockly, F., Lund, A. H. & Piechaczyk, M. Sustained systemic delivery of monoclonal antibodies by genetically modified skin fibroblasts. J Invest Dermatol 115, 740-5 (2000) and Noel, D., Dazard, J. E., Pelegrin, M., Jacquet, C. & Piechaczyk, M. Skin as a potential organ for ectopic monoclonal antibody production. J Invest Dermatol 118, 288-94 (2002) hereby incorporated by reference in its entirety.

Exemplary methods described herein utilize adeno-associated virus vectors (AAVs) which induce minimal anti-vector immunity and effectively transduce long-lived and non-replicating cells in vivo thereby providing a single-step AAV-based antibody gene transfer system to skin which doesn't require genetic engineering ex vivo and enables therapeutic enzymes, factors or proteins to be secreted to the blood stream.

Based on the present disclosure, one of skill can identify suitable vectors by testing vector constructs and determining specificity to dermal fibroblasts using an immunocompetent hairless mouse model to validated temporal expression. According to one aspect, an ultrasound-mediated gene delivery method is provided including 1) pretreating skin with ultrasound, 2) topically administering the virus to the skin followed by passive-diffusion delivery to skin cells, 3) incubation, and 4) protein quantification after tissue harvesting.

### Potency of Enzyme-Gene Transfer

According to one aspect, the viral vectors described herein transfer one or more nucleic acid sequences encoding a therapeutic enzyme, factor or protein to skin cells of a subject. The viral vectors replicate to other skin cells producing a plurality of skin cells that produce the therapeutic enzyme, factor or protein which is secreted from the skin cells and is systemically delivered to the subject. Suitable viral vectors may be identified and engineered with the assistance of an in vitro system which has low sample-to-sample variability. Three-dimensional models of human skin, also called organotypic cultures, present such a system of constant parameters related to tissue thickness, homogeneity of cell type and spatial distribution, and cell surface density.

Established models for studying penetration through stratified human skin fall into two main categories: ex vivo skin explant cultures and regenerated three-dimensional organotypic models derived from freshly isolated primary human cells. According to the present disclosure, human skin explant systems are used in a high throughput fashion to assess viral tropism, dose response and efficacy of rAAV-assisted delivery of one or more therapeutic enzymes, factors or proteins to skin cells. Skin explants provide preserved tissue morphology and presence of all resident cell types of the epidermis and dermis as well as skin appendages making them a useful system to assess potency of rAAV mediated delivery of nucleic acid encoding a therapeutic enzyme, factor or protein to fibroblasts (primary target) and keratinocytes (secondary target). An additional exemplary system is 3D cultures of in vitro reconstituted human skin equivalents. These cultures include oprimary human dermal fibroblasts and epidermal keratinocytes pooled from various adult donors. See Duperret, E. K., Oh, S. J., McNeal, A., Prouty, S. M. & Ridky, T. W. Activating FGFR3 mutations cause mild hyperplasia in human skin, but are insufficient to drive benign or malignant skin tumors. Cell Cycle 13, 1551-9 (2014); Ratushny, V., Gober, M. D., Hick, R., Ridky, T. W. & Seykora, J. T. From keratinocyte to cancer: the pathogenesis and modeling of cutaneous squamous cell carcinoma. J Clin Invest 122, 464-72 (2012); Ridky, T. W., Chow, J. M., Wong, U. J. & Khavari, P. A. Invasive three-dimensional organotypic neoplasia from multiple normal human epithelia. Nat Med 16, 1450-5 (2010); and Truong, A. B., Kretz, M., Ridky, T. W., Kimmel, R. & Khavari, P. A. p63 regulates proliferation and differentiation of developmentally mature keratinocytes. Genes Dev 20, 3185-97 (2006) each of which is hereby incorporated by reference in its entirety. Upon contact with the air, the cultures are prompted to form a full thickness multilayer model of intact human skin. The dermis and the epidermis are separated by an intact basement membrane, remain metabolically and mitotically active and thus perfectly mimic in vivo characteristics.

### Delivery to Skin Tissues

According to one aspect, the engineered viral vectors described herein are transferred into dermal and/or epidermal cells to generate durable expression and secretion of a therapeutic enzyme, factor or protein, or other biologically active polypeptides, such as HIV bnAbs in vivo. Exemplary delivery methods include topically applying the viruses described herein to the skin surface. Methods described herein include the repeated delivery of the viruses, such as rAAVs, to the skin surface of a subject. The virus may be included in a topical formulation known to those of skill in the art for application to skin. Other delivery methods known to those of skill in the art can be used to deliver the recombinant viruses to the skin. These delivery methods comprise (1) electroporation such as by applying short high voltage pulses to the skin, (2) heating the formulation as it is applied to the skin (37° C.), (3) needleless injections such as by firing liquid at supersonic speed through the stratum corneum. (4) pressure waves generated by laser radiation, fraction laser, or radiofrequency (100 kHz). (5) magnetophoresis by external magnetic field, (6) iontophoresis, (7) applying a chemical peel to the skin followed by application of the virus to the treated skin surface, (7) abrasion techniques such as diamond or sand paper abrasion, tape stripping, and the like followed by application of the virus to the treated skin surface. A person skilled in the art can choose the appropriate delivery method according to an application. These methods can be used in combination with the method of ultrasound pre-treatment of skin and administering of the recombinant viruses as disclosed herein.

According to the present disclosure, methods are provided to infect large numbers of skin cells such as fibroblasts and/or keratinocytes with the viral vectors described herein to achieve concentrations of a therapeutic enzyme, factor or protein suitable for treatment of an enzyme, factor or protein deficiency, such as associated with lysosomal storage diseases or conditions. Contrary to other delivery approaches such as intramuscular injections, the non invasive ultrasound assisted method described herein utilizes large skin areas to target cell numbers that are orders of magnitudes higher than cell numbers achievable through intramuscular injections.

According to one aspect, the rAAV viral vectors described herein provide cellular tropism and selective targeting of the one or more therapeutic enzymes, factors or proteins or other target polypeptides, such as HIV bnAbs, to dermal fibroblasts.

According to one aspect, a method is provided for rAAV-vectored gene transfer of therapeutic enzymes, factors or proteins into the skin of a mammal, such as a human. Immune-competent hairless mice (SKH-1E mouse model) are suitable models for efficacy in human skin. The skin of hairless mice is widely utilized as a substitute for human skin to measure percutaneous drug penetration in vivo. In general, hairless mouse skin is slightly more permeable than human skin but it is by far less permeable than the skin of haired mice, rats and dogs. Ultrasound or dermal micro-abrasion is used to treat the skin of the mammal. Selected rAAV serotypes carrying the foreign nucleic acids encoding the therapeutic enzyme, factor or protein are administered to the treated skin and the rAAV infects skin cells and delivers the nucleic acid sequence encoding the therapeutic enzyme, factor or protein. The infected skin cells produce the therapeutic enzyme, factor or protein which are secreted from the cells and travel into the circulatory system. Suitable dose regimens may be determined using different dose regimens applied to animals. The optimal rAAV infection dose may be determined. Cellular tropism or preferential targeting of the rAAV vectors to dermal fibroblasts (and keratinocytes) may be determined.

The practice of the disclosed methods employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Names and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### Example I

### Secretion of α-Galactosidase A (GLA) from 3D-reconstructed Skin

Aspects of the present disclosure are directed to a method of delivering the lysosomal enzyme α-galactosidase A (GLA) to an individual in need thereof such as an individual with low lysosomal enzyme α-galactosidase A (GLA) or deficient lysosomal enzyme α-galactosidase A (GLA). Such a method is referred to as enzyme replacement therapy. In this aspect, delivering the lysosomal enzyme α-galactosidase A (GLA) to an individual in need thereof is an enzyme replacement therapy for Fabry disease. Fabry disease is an X-linked deficiency of the lysosomal enzyme α-galactosidase A (GLA). GLA encodes a glycoprotein which hydrolyses ceramide trihexoside, and catalyzes the hydrolysis of melibiose into galactose and glucose. Errors in this enzyme lead to failure to process alpha-D-galactosyl glycolipids.

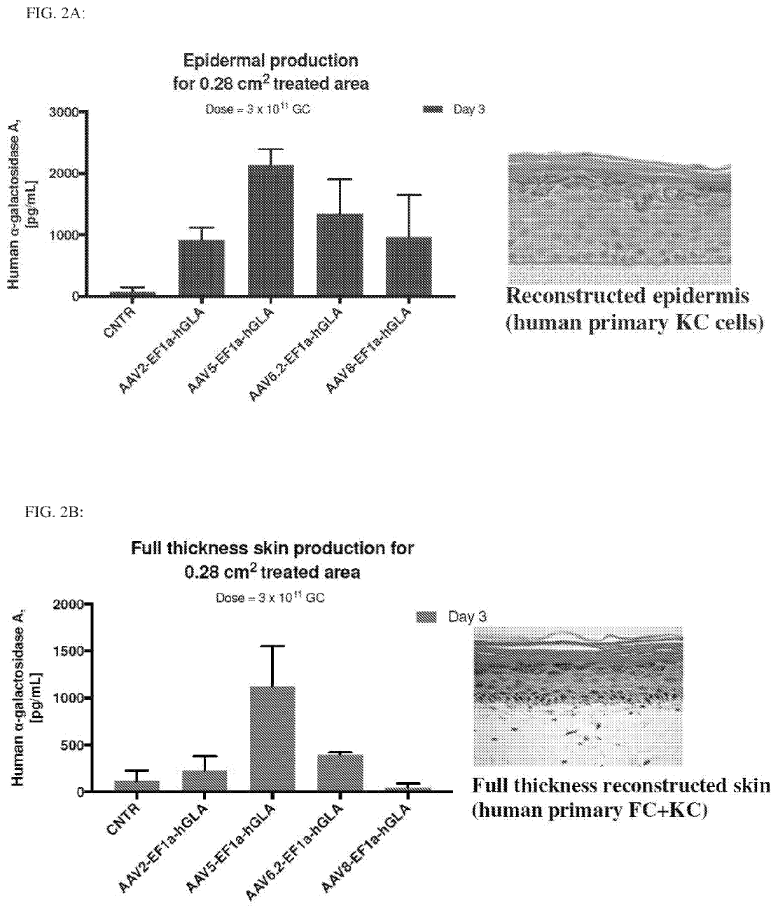

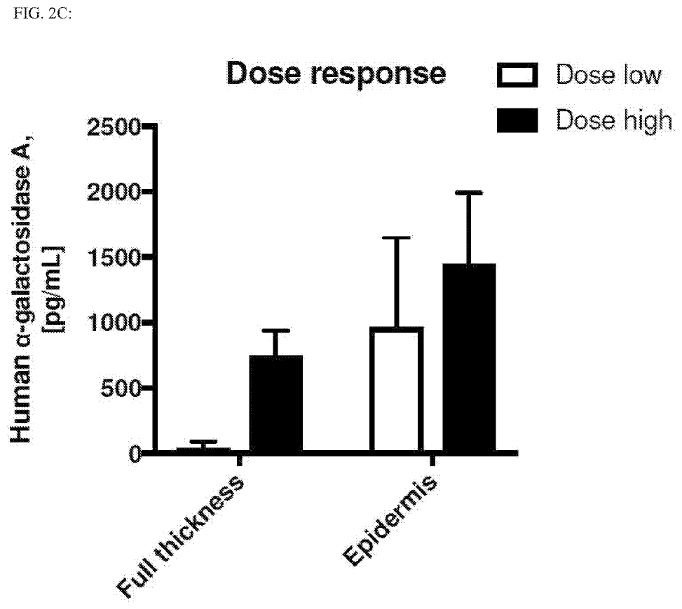

Recombinant AAV vectors were used to encode the GLA gene and deliver to skin cells for passive continuous production and systemic export to the blood stream. Validation experiments were conducted in two in vitro human models: (1) fully differentiated human epidermis; and (2) full thickness 3D-reconstructed tissue both of which are constructed from primary human cells and maintained in a transwell culture. Epidermal tissues were of 1.1 cm-diameter in a transwell format, and were treated by ultrasound at a power density of 93 W/cm² in a continuous mode for a duration of 15 s, while full thickness tissues were treated for a duration of 20 s at the same power density. The treatment surface was 0.28 cm² in all cases. Four different serotypes—AAV2, AAV5. AAV6.2, and AAV8 all driven by a ubiquitous EF1α promoter were tested in epidermis, the results of which are shown in FIG. 2A, and full thickness skin, the results of which are shown in FIG. 2B, at day 3 post-treatment in replicates of 3 tissues. Average production is given by the mean values, while error bars represent the standard error around the mean. The tested serotypes showed useful expression of human α-galactosidase A. Of the tested serotypes, AAV5 performed the best both in the epidermis and full thickness models. FIG. 2C shows dose response for two doses: high (1×10¹² GC) and low (3×10¹¹ GC) of AAV8 viral particles.

### Example II

### Tropism of Secretion in 3D-Reconstructed Tissues

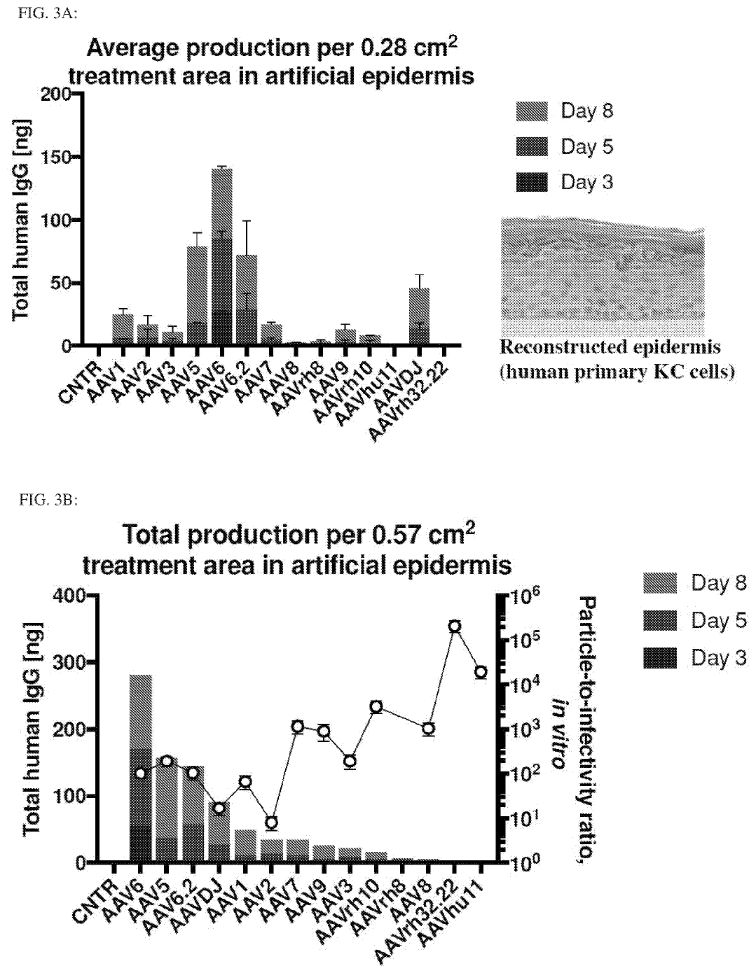

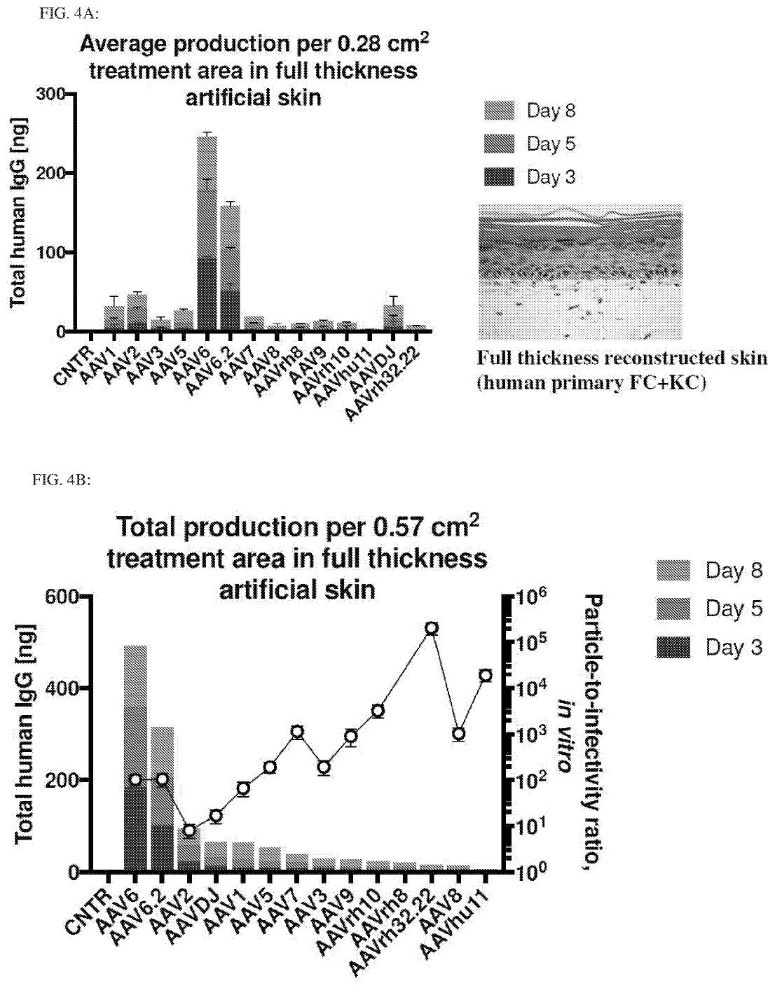

According to the present disclosure, methods are provided for improving AAV transduction to specific cell types and tissues by the use of hybrid capsids which are produced by mixing plasmids encoding capsid proteins of different serotypes during vector production. Two-fold screening of 12 known AAV hybrid capsids (AAV1, AAV2, AAV5, AAV6.2, AAV7, AAV8, AAVrh8, AAV9, AAVDJ, AAV10, AAVhu11, and AAVrh32.22) was carried out to determine optimal local transduction to skin tissues and secretion capabilities of the target cell types. Experiments were conducted in: (1) fully differentiated human epidermis (the results of which are presented in FIGS. 3A and 3B; and (2) full thickness 3D-reconstructed tissue (the results of which are presented in FIGS. 4A and 4B) maintained in a transwell culture. Hybrid AAV capsids encoding broadly neutralizing antibody bnAb (VRC01) were delivered in 2 biological replicates at a dose of 3×10¹¹ GC in 50 μl volume. Culture media was collected at days 4, 7, and 10, and analyzed by an enzyme-linked immunosorbent assay (ELISA) total human IgG levels.

As described above, epidermal tissues were treated by ultrasound at a power density of 93 W/cm² in a continuous mode for a duration of 15 s, while full thickness tissues were treated for a duration of 20 s. The surveyed serotypes were ranked by their ability to transduce cell types of highest secretion potential. As shown in FIGS. 3A and 3B and FIGS. 4A and 4B, the better performing hybrid capsids in epidermal tissues are AAV6, AAV5, and AAV6.2, while AAV6, AAV6.2 and AAV2 are the better performers in the full thickness model. Furthermore, a dose of 6×10¹¹ GC of AAV6 can produce up to 0.5 μg/mL from a treatment area of 0.56 cm² within 8 days.

In addition to tropism, particle-to-infectivity (P/I) is reported for each AAV serotype (and respective lots if more than one lot was produced) encoding bnAb (VRC01). P/I ratios are determined by the Taqman TCID50 assay based upon limiting dilution of the vector and a 50% endpoint determination of viral DNA replication using real-time PCR for sensitive, quantitative calling of positive wells. AAV vectors are serially diluted and a cell line expressing AAV rep and cap is co-infected with these dilutions plus wildtype Ad5 in a 96-well plate format (12 replicate wells per dilution). The presence of AAV rep and adenovirus helper genes allows for the replication of AAV DNA. After a suitable incubation period, DNA is extracted and a 50% endpoint determination is performed by a basic computer program based upon Karbers formula. Low P/I corresponds to higher infectious titer, while high P/I values to a low infectious titer. In FIGS. 3B and 4B, the P/I ratio in all serotype experiments demonstrate that AAV tropism of secretion is not correlated with viral infectivity.

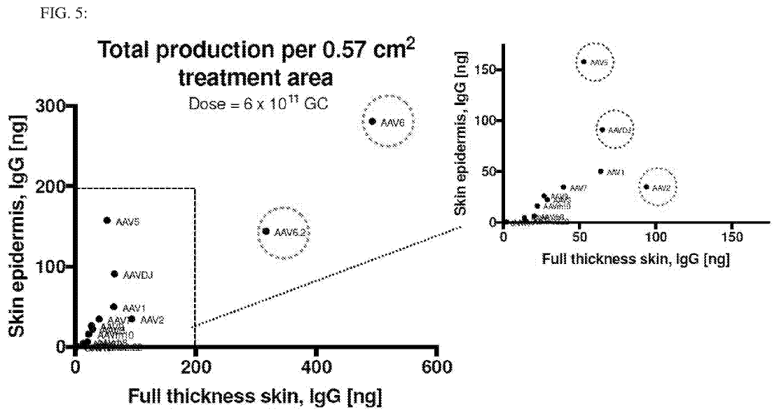

FIG. 5 represents a plot of AAV tropism of secretion in epidermal and full thickness tissues. As demonstrated, hybrid capsids of AAV6 and 6.2 have the most efficient tropism in both keratinocytes and fibroblasts while AAV5 performs well in epidermis (KC) and AAV2—in full thickness skin (FC) with minimal tropism for epidermal cells. Without wishing to be bound by scientific theory, in the skin, the tropism of the top performing AAV capsid is driven by its receptors N-linked sialic acid, and epidermal growth factor receptor (Weller et al, 2010) for AAV6, by N-linked sialic acid (Kaludov et al, 2001) and platelet derived growth factor receptor (Di Pasquale et al, 2003) for AAV5, and by heparin sulphate proteoglycan (Summerford & Samulski, 1998) and fibroblast growth factor receptor (Qing et al, 1999), for AAV2.

### Example III

### Dose Response in 3D-Reconstructed Tissues

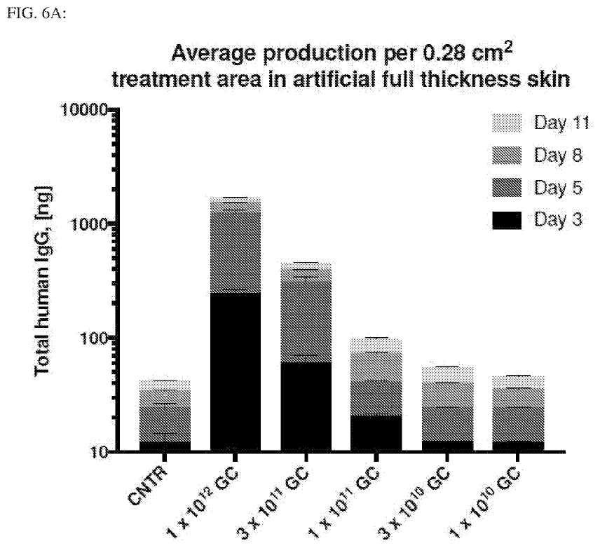

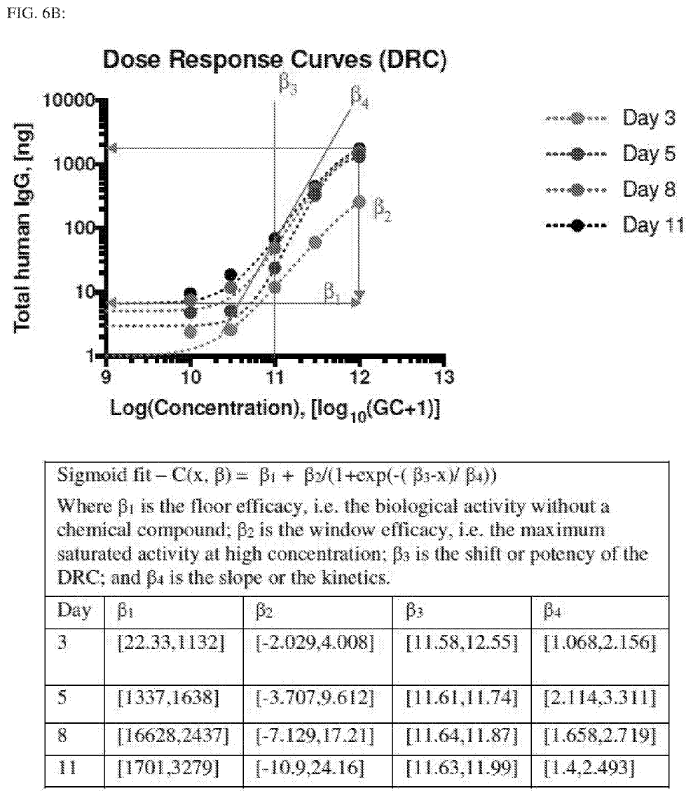

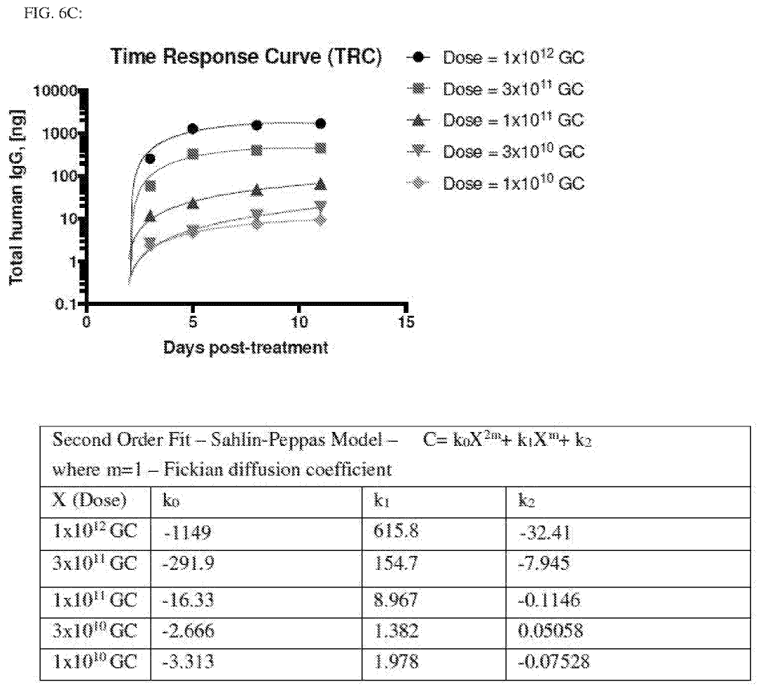

Escalation dose response was determined in 3D-reconstructed tissue models of full thickness (having both dermal and epidermal structures) for 5 doses in duplicates: 1×10¹² GC, 3×10¹¹ GC, 1×10¹¹ GC, 3×10¹⁰ GC, and 1×10¹⁰ GC. Each tissue was of 1.1 cm-diameter in a transwell format, and was treated by ultrasound at a power density of 246 W/cm² in a continuous mode for a duration of 30 s. The total cross-sectional area of treatment was 0.28 cm². The production of bnAb measured by total human IgG amounts at 4 times points (day 3, 5, 8, and 11) in culture media was used as an end point for estimates of dose response. The dose escalation data is presented on a log 10 bar plot of FIG. 6A, and exhibits a log-log response in the range from 1×10¹²2 GC through 3×10¹⁰ GC. As described in FIG. 6B, doses of therapy for secretion of systemic polypeptides should be modeled by a 4-point sigmoid curve with fitting parameters representing floor efficacy—β₁, window efficacy—β₂, maximum saturation activity at high concentration—β₃, and kinetics—β₄, as one example of determining dose response and kinetics of [production+secretions]. We quantified the DRCs at different time points (days 3, 5, 8, and 11) and show that the DRCs are higher potency after day 5. In FIG. 6C, we describe the time response curve for production of therapeutic polypeptide at the five doses described above 1×10¹² through 1×10¹⁰ GC, and propose a second order model incorporating release mechanisms based on diffusional and relaxation release mechanisms, where the reported coefficients describe non-Fickian diffusion, i.e. diffusion dependent upon concentrations of produced polypeptide (k₂), the release kinetic constant for polymer relaxation, i.e. transport through the dermal matrix (k₁), and residual release due to other factors (k₀).

### Example IV

### Secretion by Human Skin Ex Vivo

To determine the tropism of secretion data obtained in 3D-reconstructed skin, experiments were performed in human skin obtained in the form of explants from a patient who underwent abdominoplasty. Whole skin was treated by ultrasound at a power density of 246 W/cm² in a continuous mode for a duration of 30 s, and total cross-sectional area of 0.28 cm². Treated full thickness skin (without the fat) was then excised using a 1.12 cm-diameter punch biopsy, washed in PBS and cultured for 10 days in transwell format. Recombinant AAVs of 13 serotypes (AAV1, AAV2, AAV5, AAV6.2, AAV7, AAV8, AAV9, AAVDJ, AAV10, AAVhu11, AAVrh32.22, AAV-Anc80, and AAV-Anc113) were delivered in 2 biological replicates at a dose of 3×10¹¹ GC in 50 μl volume. Culture media was collected at days 4, 7, and 10, and analyzed by an enzyme-linked immunosorbent assay (ELISA) against gp-120-MN, an epitope on the encoded bnAb, VRC01.

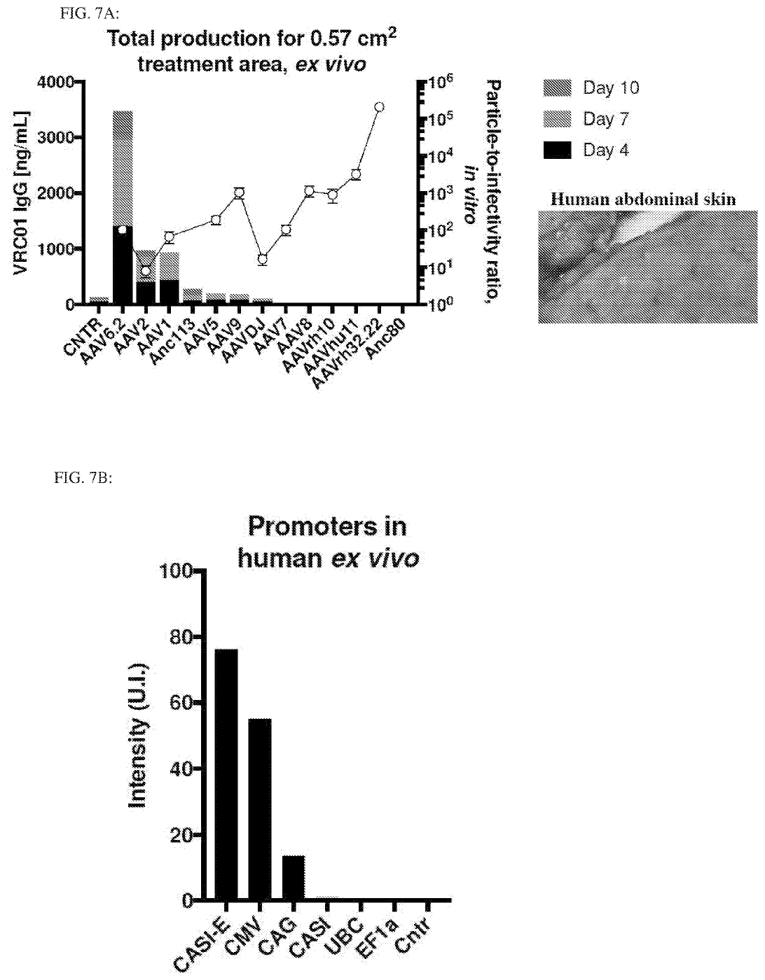

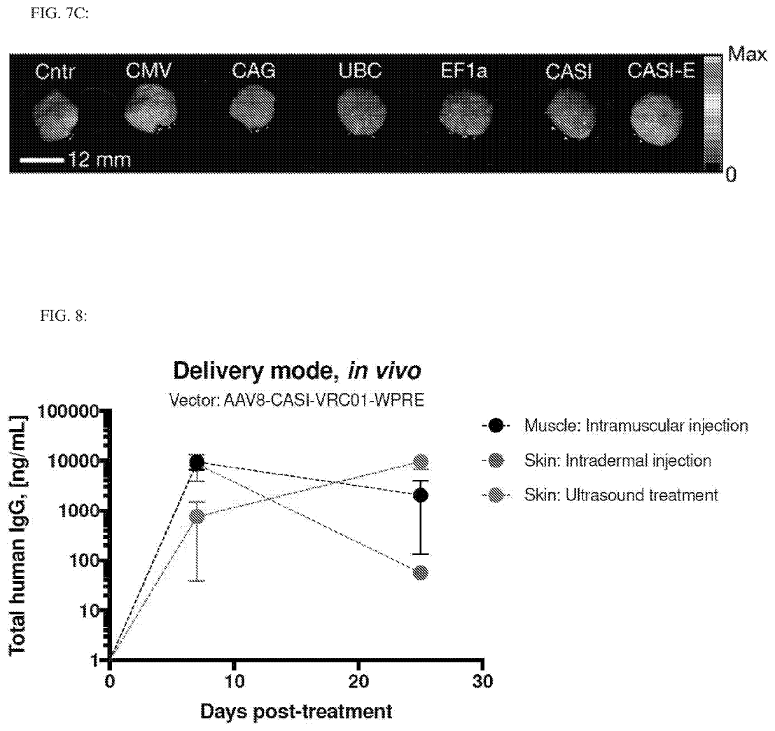

FIG. 7A shows data for amounts of bnAb secreted at three time points within 10 days (on the right axis), and in vitro particle-to-infectivity ratios (on the left axis) for the surveyed serotypes. Cumulatively, AAV6.2 produced 3.5 μg/mL from a treatment area of 0.56 cm², and performed better than other serotypes. Particle-to-infectivity ratios were determined by the TCID50 assay for the same AAV vector lots. Similar to the 3D-reconstructed skin models, no correlation was found between viral infectious titer and tropism of secretion. Next, 6 constitutive promoters (CMV, CAG, UBC, EF1a, CASI, and CASI-WPRE) driving expression of luciferase were surveyed. Ex vivo imaging of metabolically active skin explants was performed at day 10, and luciferase expression was quantified (data shown in FIG. 7B) and visualized (images shown in FIG. 7C). CASI promoter with the WPRE enhancer (CASI-E) gave the highest levels of expression.

### Example V

### Skin Treatment with rAAV Produces Sustained Long-Term Systemic Secretion

Administration of rAAV vector particles was performed in 6- to 8-week-old immunocompetent SK1H hairless mice in three delivery modes: (1) single muscle injection (in a volume of 20 μl); (2) single intradermal injection (in a volume of 20 μl), and (3) 4×0.28 cm² ultrasound treatments (in a total volume of 17 μl to account for the syringe dead volume lost in delivery modes 1 and 2. All animals received an AAV vector of serotype 8 driven by a CASI promoter, and encoding for the bnAb, VRC01 (human HIV broadly neutralizing antibody), AAV8-CASI-VRC01-WPRE-SV40. Two days prior to treatment, mice were topically administered with 0.1% dexamethasone. Blood samples were collected from the tail vein at day 7, and day 25. To separate plasma, blood samples were spun at 6000 g for 20 min. Total human IgG levels were determined by enzyme-linked immunosorbent assay (ELISA) per manufacturer's protocol.

FIG. 8 depicts data demonstrating that intramuscular and intradermal delivery modes both reach levels of ˜9.5 μg/mL at day 7, however, they decrease to 2 μg/mL and 56 ng/mL at day 25, respectively. In the case of ultrasound delivery, bnAb levels increase gradually starting from an arithmetic mean of 760 ng/mL and reaching 9.6 μg/mL at day 25. These data show successful delivery of rAAV vectors to mouse skin and sustained long-term systemic secretion. Delivery by ultrasound outperforms both intramuscular and intradermal modes in the long run. Because mouse dermal fibroblasts have a cell cycle of 9-12 months, skin is used for administration of enzyme replacement therapy once every 12 months.

### Example VI

### Embodiments

Aspects of the present disclosure are directed to a method of systemic delivery of an enzyme or factor to an enzyme or factor deficient subject in need thereof including genetically modifying target skin cells within skin of the subject by administering to the subject an engineered virus comprising one or more foreign nucleic acid sequences encoding the enzyme or factor deficient in the subject to treat a lysosomal storage disease, wherein the one or more foreign nucleic acid sequences of the engineered virus are introduced into the target skin cells within the skin to produce genetically modified skin cells, and wherein the genetically modified skin cells produce the enzyme or factor deficient in the subject by expression of the one or more foreign nucleic acid sequences, and wherein the enzyme or factor is excreted from the genetically modified skin cells and is introduced systemically within the subject in an amount sufficient to treat deficiency of the enzyme or factor in the subject by raising the amount of the enzyme or factor within the subject. According to one aspect, the engineered virus is transmitted in vivo between target skin cells to create additional genetically modified skin cells producing the enzyme or factor deficient in the subject. According to one aspect, the administering of the engineered virus comprises topically applying a formulation comprising the engineered virus to skin of the subject. According to one aspect, the genetically modified skin cells are long-lived and non-replicating. According to one aspect, the enzyme or factor is a member selected from the group consisting of α-galactosidase A (GLA), α-galactosidase B, β-galactosidase (GLB1), neuraminidase 1 (NEU1), glucocerebrosidase, ceramidase (ASAH1), beta-hexosaminidase, hexosaminidase A, hexosaminidase B, sphingomyelinase, sulphatase, galactocerebrosidase, lysosomal acid lipase (LAL), glucocerebrosidase, arylsulfatase A (ARSA), arylsulfatase B (ARSB), formylglycine-generating enzyme (FGE), α-L-iduronidase, iduronidase, iduronate sulfatase, iduronate-2-sulfatase (12S), heparan sulfamidase, n-acetylglucosaminidase, heparan-α-glucosaminide, N-acetyltransferase, acetyltransferase, N-acetylglucosamine-6-sulfatase, galactose-6-sulfate sulfatase, N-acetylgalactosamine-4-sulfatase, galactosamine-6-sulfate sulfatase, β-glucuronidase, hyaluronidase, HYAL1, HYAL2, HYAL3, HYAL4, HYAL5, SPAM1, PH-20, HYAL6, HYALP1, hyaluronoglucosidase, hyauronoglucuronidase, cathepsin A, glycosidase, α-N-acetyl neuraminidase (sialidase), phosphotransferase, mucolipid1, palmitoyl-protein thioesterase, tripeptidyl peptidase, PPT1, TPP1, α-D-mannosidase, beta-mannosidae, aspartylglucosaminidase, alpha-L-fucosidae, alpha-glucosidase, cystinosin, cathepsin K, sialin, solute carrier family 17, and prosaposin. According to one aspect, the engineered virus is a genetically modified virus. According to one aspect, the engineered virus is a non-integrative viral vector. According to one aspect, the engineered virus is an adeno-associated viral vector. According to one aspect, the lysosomal storage disease is Fabry disease. According to one aspect, the enzyme is α-galactosidase A and the genetically modified skin cells produce the α-galactosidase A over a sustained period of time. According to one aspect, the enzyme is α-galactosidase A and is introduced systemically within the subject by introduction into a circulatory system of the subject. According to one aspect, the subject is a mammal. According to one aspect, the subject is a human. According to one aspect, the skin cells are human skin cells. According to one aspect, the skin is treated to be permeabilized to the engineered virus. According to one aspect, stratum corneum of the skin is processed to be permeabilized to the engineered virus. According to one aspect, the skin is pretreated with cavitational ultrasound or microdermabrasion to disrupt the cutaneous stratum corneum, and wherein the engineered virus is transported to the epidermis, the papillary and reticulous dermis. According to one aspect, the skin cells are dermal fibroblast cells or epidermal progenitor cells. According to one aspect, the skin is treated with ultrasound prior to administering the engineered virus. According to one aspect, the skin is treated with ultrasound prior to administering the recombinant virus and ultrasound is stopped prior to administering the engineered virus. According to one aspect, the skin is treated with ultrasound at a frequency between about 10 kHz and about 100 kHz or about 10 kHz and about 20 kHz. According to one aspect, the skin is treated with ultrasound applied at an intensity between about 1 W/cm² and about 10 W/cm² or about 1 W/cm² and about 300 W/cm². According to one aspect, the skin is treated with ultrasound applied for a duration between about one minute to about 10 minutes. According to one aspect, the skin is treated with ultrasound applied continuously or at duty cycles in the range of between 20% and 100%. According to one aspect, the skin is treated with ultrasound applied topically or intra-dermally. According to one aspect, the engineered virus is a retrovirus, adenovirus, adeno-associated virus (AAV), vaccinia virus or herpes simplex virus. According to one aspect, the engineered virus is a recombinant AAV of serotype 1, 2, 3, 4, 5, 6, 6.2, 7, 8, 9, DJ, 10, hu11, rh32.22, Anc80 or Anc113. According to one aspect, the engineered virus is applied to skin once weekly. According to one aspect, the engineered virus is applied to skin once monthly. According to one aspect, the engineered virus is applied to skin once yearly. According to one aspect, the skin cells are dermal fibroblasts or keratinocytes. According to one aspect, the skin cells are dermis skin cells and the engineered virus is a recombinant AAV of serotype 2, 6, or 6.2. According to one aspect, the skin cells are epidermis skin cells and the engineered virus is a recombinant AAV of serotype 5, 6 or 6.2. According to one aspect, the dose of virus is 3×10¹¹ GC or greater. According to one aspect, the skin cells are dermis skin cells and the engineered virus is a recombinant AAV of serotype 2, 6, or 6.2, and wherein the dose of virus is 3×10¹¹ GC or greater. According to one aspect, the skin cells are epidermis skin cells and the engineered virus is a recombinant AAV of serotype 5, 6 or 6.2, and wherein the dose of virus is 3×10¹¹ GC or greater.

### OTHER EMBODIMENTS

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference in their entirety for all purposes to the same extent as if each individual publication or patent application were specifically and individually indicated to be so incorporated by reference.

1. A method of systemic delivery of an enzyme or factor to an enzyme or factor deficient subject in need thereof comprising
genetically modifying target skin cells within skin of the subject by administering to the subject an engineered virus comprising one or more foreign nucleic acid sequences encoding the enzyme or factor deficient in the subject to treat a lysosomal storage disease,
wherein the one or more foreign nucleic acid sequences of the engineered virus are introduced into the target skin cells within the skin to produce genetically modified skin cells, and wherein the genetically modified skin cells produce the enzyme or factor deficient in the subject by expression of the one or more foreign nucleic acid sequences, and
wherein the enzyme or factor is excreted from the genetically modified skin cells and is introduced systemically within the subject in an amount sufficient to treat deficiency of the enzyme or factor in the subject by raising the amount of the enzyme or factor within the subject.

2. The method of claim 1 wherein the engineered virus is transmitted in vivo between target skin cells to create additional genetically modified skin cells producing the enzyme or factor deficient in the subject.

3. The method of claim 1 wherein the administering of the engineered virus comprises topically applying a formulation comprising the engineered virus to skin of the subject.

4. The method of claim 1 wherein the genetically modified skin cells are long-lived and non-replicating.

5. The method of claim 1 wherein the enzyme or factor is a member selected from the group consisting of α-galactosidase A (GLA), α-galactosidase B, β-galactosidase (GLB1), neuraminidase 1 (NEU1), glucocerebrosidase, ceramidase (ASAH1), beta-hexosaminidase, hexosaminidase A, hexosaminidase B, sphingomyelinase, sulphatase, galactocerebrosidase, lysosomal acid lipase (LAL), glucocerebrosidase, arylsulfatase A (ARSA), arylsulfatase B (ARSB), formylglycine-generating enzyme (FGE), α-L-iduronidase, iduronidase, iduronate sulfatase, iduronate-2-sulfatase (I2S), heparan sulfamidase, n-acetylglucosaminidase, heparan-α-glucosaminide, N-acetyltransferase, acetyltransferase, N-acetylglucosamine-6-sulfatase, galactose-6-sulfate sulfatase, N-acetylgalactosamine-4-sulfatase, galactosamine-6-sulfate sulfatase, β-glucuronidase, hyaluronidase, HYAL1, HYAL2, HYAL3, HYAL4, HYAL5, SPAM1, PH-20, HYAL6, HYALP1, hyaluronoglucosidase, hyauronoglucuronidase, cathepsin A, glycosidase, α-N-acetyl neuraminidase (sialidase), phosphotransferase, mucolipid1, palmitoyl-protein thioesterase, tripeptidyl peptidase, PPT1, TPP1, α-D-mannosidase, beta-mannosidase, aspartylglucosaminidase, alpha-L-fucosidase, alpha-glucosidase, cystinosin, cathepsin K, sialin, solute carrier family 17, and prosaposin.

6. The method of claim 1 wherein the engineered virus is a genetically modified virus.

7. The method of claim 1 wherein the engineered virus is a non-integrative viral vector.

8. The method of claim 1 wherein the engineered virus is an adeno-associated viral vector.

9. The method of claim 1 wherein the lysosomal storage disease is Fabry disease.

10. The method of claim 1 wherein the enzyme is α-galactosidase A and the genetically modified skin cells produce the α-galactosidase A over a sustained period of time.

11. The method of claim 1 wherein the enzyme is α-galactosidase A and is introduced systemically within the subject by introduction into a circulatory system of the subject.

12. The method of claim 1 wherein the subject is a mammal.

13. The method of claim 1 wherein the subject is a human.

14. The method of claim 1 wherein the skin cells are human skin cells.

15. The method of claim 1 wherein the skin is treated to be permeabilized to the engineered virus.

16. The method of claim 1 wherein stratum corneum of the skin is processed to be permeabilized to the engineered virus.

17. The method of claim 1 wherein the skin is pretreated with cavitational ultrasound or microdermabrasion to disrupt the cutaneous stratum corneum, and wherein the engineered virus is transported to the epidermis, the papillary and reticulous dermis.

18. The method of claim 1 wherein the skin cells are dermal fibroblast cells or epidermal progenitor cells.

19. The method of claim 1 wherein the skin is treated with ultrasound prior to administering the engineered virus.

20. The method of claim 1 wherein the skin is treated with ultrasound prior to administering the recombinant virus and ultrasound is stopped prior to administering the engineered virus.

21. The method of claim 1 wherein the skin is treated with ultrasound at a frequency between about 10 kHz and about 100 kHz or about 10 kHz and about 20 kHz.

22. The method of claim 1 the skin is treated with ultrasound applied at an intensity between about 1 W/cm² and about 10 W/cm² or about 1 W/cm² and about 300 W/cm².

23. The method of claim 1 wherein the skin is treated with ultrasound applied for a duration between about one minute to about 10 minutes.

24. The method of claim 1 wherein the skin is treated with ultrasound applied continuously or at duty cycles in the range of between 20% and 100%.

25. The method of claim 1 wherein the skin is treated with ultrasound applied topically or intra-dermally.

26. The method of claim 1 wherein the engineered virus is a retrovirus, adenovirus, adeno-associated virus (AAV), vaccinia virus or herpes simplex virus.

27. The method of claim 1 wherein the engineered virus is a recombinant AAV of serotype 1, 2, 3, 4, 5, 6, 6.2, 7, 8, 9, DJ, 10, hu11, rh32.22, Anc80 or Anc113.

28. The method of claim 1 where the engineered virus is applied to skin once weekly.

29. The method of claim 1 where the engineered virus is applied to skin once monthly.

30. The method of claim 1 where the engineered virus is applied to skin once yearly.

31. The method of claim 1 wherein the skin cells are dermal fibroblasts or keratinocytes.

32. The method of claim 1 wherein the skin cells are dermis skin cells and the engineered virus is a recombinant AAV of serotype 2, 6, or 6.2.

33. The method of claim 1 wherein the skin cells are epidermis skin cells and the engineered virus is a recombinant AAV of serotype 5, 6 or 6.2.

34. The method of claim 1 wherein the dose of virus is 3×10¹¹ GC or greater.

35. The method of claim 1 wherein the skin cells are dermis skin cells and the engineered virus is a recombinant AAV of serotype 2, 6, or 6.2, and wherein the dose of virus is 3×10¹¹ GC or greater.

36. The method of claim 1 wherein the skin cells are epidermis skin cells and the engineered virus is a recombinant AAV of serotype 5, 6 or 6.2, and wherein the dose of virus is 3×10¹¹ GC or greater.
